(19) United States
(12) Patent Application Publication
(10) Pub. No.: US 2024/0158317 A1
(43) Pub. Date: May 16, 2024

(54) Fuel Additive

(71) Applicant: Kendel Covington, Portland, OR (US)

(72) Inventor: Kendel Covington, Portland, OR (US)

(21) Appl. No.: 18/281,756

(22) PCT Filed: Mar. 11, 2022
(86) PCT No.: PCT/US2022/020060

§ 371 Date: Sep. 12, 2023

(51) Int. Cl. C07C 2/82; C10L 1/16; C10L 10/04

(52) U.S. Cl. CPC C07C 2/82; C10L 1/1608; C10L 10/04; C10L 2230/22

(57) ABSTRACT

A method of producing a fuel additive which uses hydrocarbon molecules to rearrange the molecules of a straight chain alkane, with the loss of a hydrogen molecule. This process enables straight chain alkanes to be converted into branched-chain alkanes, cyclohexenes, and aromatic hydrocarbons which are used to enhance gas and diesel fuels. The additive produced by the process reduces emissions, increases power, and increases efficiency.

### 1. FIELD OF THE INVENTION

The present invention generally relates to fuel additives. More specifically, the present invention relates to a method of making an improved fuel additive.

### BACKGROUND OF THE INVENTION

Fuel additives for increasing fuel efficiency are well known. Manufacturers such as STP and Chevron have been producing fuel additives for years with varying degrees of success. A major problem with known fuel additives is that while somewhat effective at reducing deposits in fuel injectors and other parts of the engine and exhaust, they are not very effective at increasing fuel efficiency. Also, 10 to 12 ounces of additive are needed for this effect, adding as much as 20 dollars (US) to the cost of refueling.

Accordingly, it is desirable to provide a fuel additive that is effective at both reducing unburnt hydrocarbon in the exhaust and producing higher engine temperature which burns carbon deposits in the engine, resulting in fewer deposits. Also, mixing of fuel additive with fuel is enhanced, such that a small amount of the inventive additive may be added at any time, not just when refueling.

### SUMMARY OF THE INVENTION

It is a major object of the invention to provide an improved fuel additive.

It is another object of the invention to provide an improved fuel additive that increases fuel efficiency.

It is another object of the invention to provide an improved fuel additive that can reduce engine and exhaust deposits.

It is another object of the invention to provide an improved fuel additive that is cost effective.

It is another object of the invention to provide an improved fuel additive having high solubility.

Accordingly, it is one object of the present invention to provide an additive for carrier fuels which will enable the user to fuel an internal combustion engine and enhance mileage, power output,

### BRIEF DESCRIPTION OF THE DRAWINGS

FIG. 1 is an illustration of a chemical reaction used to create the fuel additive of the invention.

### DETAILED DESCRIPTION

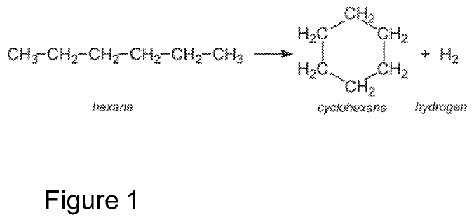

The inventive fuel additive may be produced from any straight chain alkane such as pentane, methane, butane, or hexane. FIG. 1 shows a chemical reaction leading to a fuel additive formed in accordance with the invention. The inventive process uses hydrocarbon molecules to rearrange the molecules of the straight chain alkane, with the loss of a hydrogen molecule. The reaction may be brought about by any well known alkene forming reaction as would be apparent to one of skill in the art. This process enables straight chain alkanes to be converted into branched-chain alkanes, cyclohexenes, and aromatic hydrocarbons, thus forming the inventive additive. The additive is used to enhance gas and diesel fuels used to run engines for vehicles, generators, etc. The conversion of an alkane molecule into a cycloalkane allows for a complete chain action or re-action within other hydrocarbons such as gasoline or diesel fuel, the chain reaction occurring as a result of adding the additive to a quantity of fuel in a predetermined ratio as described below. The reaction shown in FIG. 1 can be accomplished using well known methods that are apparent to one of skill in the art, and any straight chain alkane may be used in place of the hexane. Lubricity components or friction modifiers are added to the additive created by the above described process. Any combination of organic friction modifiers (OFMs), oil-soluble organo-molybdenum additives, functionalized polymers, or dispersed nanoparticles may be used for this purpose, in a percentage of less than 5% or as would be apparent to one of skill in the art based on the particular substance used.

Gasoline is a complex combination of relatively volatile hydrocarbons (typically C4 to C12) with or without small quantities of additives, blended to form a fuel suitable for use in spark ignition internal combustion engines. “Motor gasoline”, as defined in the American Society for Testing and Materials ASTM Specification D 4814 or Federal Specification VV-G-1690C, is characterized as having a boiling range of 122 degrees F. to 158 degrees F. at the 10-percent recovery point to 365 degrees F. to 374 degrees F. at the 90-percent recovery point. Motor gasoline includes conventional gasoline, reformulated gasoline, oxygenated gasoline including gasohol, and other finished gasoline. For the purpose of the present invention the term “gasoline” refers to any liquid fuel that can be used to operate a spark ignition internal combustion engine.

Diesel fuel is also a complex combination of relatively volatile hydrocarbons (typically C12 to C30) with or without small quantities of additives and is typically characterized by having a boiling range of approximately 340 degrees F. to 650 degrees F. As the inventive compound can be used to enhance hydrocarbons, it can be used in both gas and diesel fuel.

The fuel additive of the present invention is a mixture added to a carrier fuel such as gasoline or diesel which increases power output and increases mileage, as well as providing other benefits as discussed below. The lubricity components or friction modifiers may be added to the fuel additive so that it can provide upper cylinder lubrication.

The fuel additive decreases exhaust emissions an average of 87%. Test groups reported significant increases in fuel mileage as discussed below. The fuel additive functions as a fuel stabilizer, an anti-gel additive, fuel system cleaner, and an upper cylinder lubricant. The inventive fuel additive uses no alcohol. The fuel additive works in spark ignition or diesel-based fuels to radically improve the oxidation reaction of a fuel source for an overall better burn. This results in more thermal energy and fewer harmful by-products.

In use, the user will introduce the inventive fuel additive into the gas tank at a ratio of one ounce of the additive to twenty-five gallons of gasoline or one ounce of additive to twenty-five gallons of diesel. The introduction of the additive to the in& system will induce a chemical reaction in the fuel to make it more readily combustible without agitation. The action results in reduced unburnt hydrocarbon in the exhaust and higher engine temperature which burns carbon deposits in the engine. This leads to higher fuel efficiency and lower emissions from the exhaust.

Samples of the inventive fuel additive were tested to determine the effectiveness of the additive for enhancing mileage, reducing emissions, and increasing power output when added to diesel fuel. The test was conducted using JOHN DEERE PVX 6.8, JOHN DEERE POWER TECH 4.5 L, and DETROIT DIESEL 60P 11.1 L industrial engines.

The inventive additive reduced load stress by 24%. The lubricity components allowed generators to run more smoothly, with no complications during start-ups. An operational ratio of 1 oz of the inventive fuel additive to 25 gallons of fuel was used during the testing. The ratio was perfectly maintained during the testing period. This ratio is required to obtain proper test data. In previous testing not associated with this test, adding more than the specified 1:25 ratio resulted in increased fuel consumption. Fuel economy showed a 20.40% improvement resulting in an apparent fuel cost per gallon reduction from $3.72 to $2.83 per gallon. Actual cost of the inventive additive is 9 cents per treated gallon. Total actual cost per treated gallon of fuel consumed decreased to $2.92 (as opposed to the starting fuel price of $3.72). Thus, the overall fuel savings is 80 cents per treated gallon. It should be noted that the price of the fuel and the instant additive are transient and relative, the numbers used merely to illustrate increased efficiency. The testing procedure was empirical in design and conducted by the site operator. The collective fuel consumption (for all engines) read 108 gallons per day over a 30-day period.

Implementation of the inventive treatment reduced consumption to 86 gallons per day for an average daily savings of 22 gallons of fuel per day. All three power plants were fueled through a shared 1000-gallon fuel tank.

I claim:

1. A method of producing a fuel additive comprising the steps of:
converting a straight chain alkane into a branched chain alkane.

2. The method of claim 1 where the straight chain alkane is hexane.

3. The method of claim 1 where the straight chain alkane is pentane.

4. The method of claim 1 where the straight chain alkane is butane.

5. The method of claim 1 where the straight chain alkane is methane.

6. The method of claim 1 where the branched chain alkane is cyclohexane.
